(19) United States
(12) Patent Application Publication
(10) Pub. No.: US 2025/0034797 A1
(43) Pub. Date: Jan. 30, 2025

(54) METHOD FOR OBTAINING ANTIMICROBIALLY ACTIVE NONWOVEN FABRIC

(71) Applicants: BIOVALLEY SPÓLDZIELNIA SOCJALNA, Warszawa (PL); PLITECHNIKA LÓDZKA, Lódz (PL)

(72) Inventors: Anna MARZEC, Lódz (PL); Boleslaw SZADKOWSKI, Lódz (PL); Malgorzata HALLER, Warszawa (PL)

(73) Assignees: BIOVALLEY SPÓLDZIELNIA SOCJALNA, Warszawa (PL); PLITECHNIKA LÓDZKA, Lódz (PL)

(21) Appl. No.: 18/714,153

(22) PCT Filed: Dec. 2, 2022
(86) PCT No.: PCT/PL2022/050087

§ 371 Date: May 29, 2024

(30) Foreign Application Priority Data
Dec. 3, 2021 (PL) P.439740

(51) Int. Cl. D06M 11/44; D06M 13/123; D06M 13/207; D06M 15/03; D06M 15/71; D06M 16/00

(52) U.S. Cl. CPC D06M 11/44; D06M 13/123; D06M 13/207; D06M 15/03; D06M 15/71; D06M 16/00

(57) ABSTRACT

The subject of the invention is a method of obtaining an antimicrobially active nonwoven fabric, which is prepared in an aqueous solution containing: • from 0.1 to 30 wt % citric acid, • from 0.1 to 20 wt % chitosan, • from 0.1 to 15 wt % zinc oxide, • from 0.01 to 3 wt % lawsone, and • and preferably natural essential oils in an amount of 0.01 to 5 wt %, especially of mint or lavender. In the next step, the prepared solution is applied to the non-woven fabric, which is then dried.

### TECHNICAL FIELD

The subject of the invention is a method of obtaining a nonwoven fabric that is antimicrobially active against both bacteria and fungi. The nonwoven fabric obtained by the invented method is intended especially for disposable clothing for medical personnel, as well as for pajamas, bedding or underwear used in health care centers.

### BACKGROUND ART

The patent literature contains descriptions of several methods of obtaining nonwoven fabrics for the production of clothing and dressings with biostatic properties. The highly water-absorbent and breathable medical non-woven fabric in patent CN201810815169A contains modified polyvinyl alcohol fiber, modified mulberry fiber, modified chitosan fiber, carboxymethylcellulose, casein, aloe fiber, sodium alginate, starch, polyvinylpyrrolidone, and plant extract.

Patent PL229041B1 describes a method of applying an antibacterial coating (zinc oxide nanoparticles) to a knitted fabric or other fabric, to produce textiles characterized by high microbiological activity, e.g. against colibacillosis and Staphylococcus aureus.

In patent CN202110017875A, a method is described for obtaining non-woven masks incorporating antibacterial substances, such as honeysuckle extract, chitin, chitosan, or calcium carbonate.

In general, the biocidal properties of compounds such as citric acid (https://doi.org/10.1111/lam.13420), zinc oxide, chitosan, and lawsone (https://doi.org/10.1016/j.medmal) are known from prior studies art. (2012.05.002). These compounds can even be used as ingredients in cosmetics intended for skin care and hair (US2004219124A1). However, there no technologies have been developed for the production of nonwovens that incorporate these compounds using technologically uncomplicated processes and without negatively affecting the mechanical and aesthetic properties of the nonwovens. Common problems when applying coatings with biocidal properties on polymer fibers include low adhesion, deterioration of the mechanical properties (flexibility) of the nonwovens, unsightly final visual effects, and the complexity of the physical and chemical procedures used. At the same time, biocidal effectiveness is limited to certain strains of bacteria or fungi.

### SUMMARY OF INVENTION

### Technical Problem

The aim of the invention was to obtain non-woven fabrics intended for disposable medical clothing with properties limiting the growth of bacteria and fungi (antimicrobially active non-woven fabric) and characterized by good functional and aesthetic properties. The method of manufacturing the non-woven fabric is technologically uncomplicated. The nonwovens are characterized by a broad spectrum of antimicrobial activity (for at least two strains of bacteria and fungi, and even viruses).

### Solution to Problem

The essence of the invention is a method of obtaining an active non-woven antimicrobial fabric which is prepared in an aqueous solution containing: (i) 0.1-30 wt. % citric acid; (ii) 0.1-20 wt. % chitosan; (iii) 0.1-15 wt. % zinc oxide; (iv) 0.01-3 wt. % lawsone. This solution is applied to the non-woven fabric, which is then dried.

The preferred procedure is to first add citric acid to the water, then once it is dissolved, to add chitosan, followed by zinc oxide and lawsone.

Optionally, chitosan is dissolved in an aqueous solution of citric acid at a temperature of at least 40° C., then zinc oxide and lawsone are introduced at a temperature of at least 50° C., with intensive mixing and homogenization of the mixture after each ingredient is introduced.

The solution is applied to the non-woven fabric by immersing it in the prepared solution at a temperature of at least 30° C. for at least 15 min.

Finally, the nonwoven fabric is extracted from the solution and dried at a temperature of at least 40° C. for at least 20 minutes.

The solution may additionally contain 0.01-5 wt % essential oils of plant origin, especially mint or lavender. Essential oils should be introduced last, before the solution is applied to the non-woven fabric.

### Advantageous Effects of Invention

The advantage of the invented method is its use of simple technology for its implementation and natural components in proportions that can impart good or very good antimicrobial activity to the nonwoven fabric, against both bacteria and fungi. The nonwoven fabric obtained according to the invented method effectively reduces the development not only of bacteria and fungi but also of viruses, thus helping to prevent the transmission of diseases and infections. In addition, the nonwoven fabric produced by the invented method is characterized by good functional and aesthetic properties. The mechanical properties of the nonwoven fabric do not deteriorate, while its color is intensified. The nonwovens produced by the invented method are characterized by a broad spectrum of biocidal activity, as confirmed experimentally for two strains of bacteria and two strains of fungi. The incorporation of natural essential oils imparts fragrance to the non-woven fabric while providing at the same time good antimicrobial properties.

### DESCRIPTION OF EMBODIMENTS

The method according to the invention in the embodiments proceeds in three main stages:
1) Preparation of a solution consisting of:
water,
citric acid (2-hydroxypropane-1,2,3-tricarboxylic acid),
chitosan (with a molecular weight of 310,000-375,000 Da),
zinc oxide (in the form of nanometric powder),
lawsone (2-hydroxy-1,4-naphthoquinone),
peppermint or lavender oil.
2) Applying the prepared solution to the polypropylene-viscose non-woven fabric by immersing it in the solution.
3) Drying the non-woven fabric.

The compositions of the solutions used are listed in Table 1.

TABLE 1
The composition of the solution used in the embodiments
of the method according to the invention

| | Concentration | First preferred | Second preferred |
|---|---|---|---|
| Component | range (wt %) | shares (wt %) | shares (wt %) |
| Water | in the remaining | 83.6 | 85.3 |
| | range | | |
| Citric acid | 0.1-30 | 12.5 | 10.2 |
| Chitosan | 0.1-20 | 2.5 | 3.4 |
| Zinc oxide | 0.01-15 | 0.8 | 0.1 |
| Lawsone | 0.01-3 | 0.2 | 0.1 |
| Peppermint or | 0.01-5 | 0.4 | 0.9 |
| lavender oil | | | |

### Preferred Example No. 1

Into a beaker equipped with a magnetic stirrer, thermometer, and heating system were added 100 ml of water and 15 g of citric acid. The solution was stirred for 15 min, then heated to 70° C. Next, 3 g of chitosan was added while stirring. The reaction was carried out for 20 min. While stirring at 70° C., 1 g of zinc oxide and 0.2 g of lawsone were added. The reaction was continued at this temperature for 30 min.

After cooling the solution to 30° C., 0.5 g of peppermint oil was added. The solution was mixed. The non-woven fabric was then immersed in the solution for 20 min. In another variant of the invented method, instead of dipping the solution was sprayed onto both sides of the nonwoven fabric.

In the last step, after the application of the solution, the non-woven fabric was dried at 40° C. for 20 min.

The obtained nonwoven fabric showed very good antimicrobial activity against bacteria and fungi (Table 2).

TABLE 2
Biocidal activity against E. coli and S. aureus and
fungi C. albicans and A. niger. Tests were performed
in accordance with PN-EN standards ISO 20645: 2006
(bacteria) and PN-EN ISO 14119: 2005 (fungi)
Biocidal activity

| E. coli | S. aureus | C. albicans | A. niger |
|---|---|---|---|
| +++ | +++ | +++ | +++ |

Very good activity +++,
Good activity ++,
Low activity +,
No activity −

### Preferred Example No. 2

Into a beaker equipped with a magnetic stirrer, thermometer, and heating system were added 100 ml of water and 12 g of citric acid. The solution was stirred for 5 min, then heated to 40° C. Next, 4 g of chitosan was added with stirring. The reaction was carried out for 40 min. While stirring at 50° C., 0.1 g of zinc oxide and 0.06 g of lawsone were added. The reaction was continued at this temperature for 30 min.

After cooling the solution to 40° C., 1 g of lavender oil was added. The solution was mixed. The non-woven fabric was then immersed in the solution for 15 minutes. In another variant of the invented method, instead of dipping the solution was sprayed on both sides of the nonwoven fabric.

Finally, the non-woven fabric was dried at 40° C. for 120 min. The obtained nonwoven fabric showed very good antimicrobial activity against bacteria and very good or good activity against fungi (Table 3).

TABLE 3
Biocidal activity against E. coli and S. aureus bacteria
and C. albicans and A. niger fungi. Tests were performed
in accordance with PN-EN ISO 20645: 2006 (bacteria) and
PN-EN ISO 14119: 2005 standards (fungi)
Biocidal activity

| E. coli | S. aureus | C. albicans | A. niger |
|---|---|---|---|
| +++ | +++ | +++ | ++ |

Very good activity +++,
Good activity ++,
Low activity +,
No activity −

The obtained fabrics showed surprisingly good antiviral activity (more than 99% reduction of virus load) in experimental tests using the human influenza A virus (Human influenza virus A/H1N1 strain A/PR/8/34; ATCC-VR-1469™, Orthomyxoviridae). Table 4 shows the antiviral activity against A/H1N1 (n=9) of materials produced according to the method described in Preferred example No. 1 (T1 fabric) and Preferred example No. 2 (T2 fabric).

TABLE 4
Antiviral activity of the tested materials against A/H1N1 (n = 9)

| | Virus titer after 2 h | Virus titer | Virus titer |
|---|---|---|---|
| | incubation | reduction | reduction |
| Fabric | logTCID₅₀ | logTCID₅₀ | % |
| T1 | 1.72 | 2 | 99.00 |
| T2 | 0.5 | 3.2 | 99.93 |
| Reference | 3.72 | — | — |
| A/H1N1 (control) | 3.83 | — | — |

The tests were performed in accordance with ISO 18184. The reduction in viral load caused by the test materials was calculated using the Spearman-Karber method.

1. The method of obtaining antimicrobially active nonwoven fabric is characterized in that it uses an aqueous solution comprising:
from 0.1 to 30 wt % citric acid,
from 0.1 to 20 wt % chitosan,
from 0.1 to 15 wt % zinc oxide,
from 0.01 to 3 wt % lawsone, and
the solution is applied to a non-woven fabric, which is then dried.

2. The method according to claim 1, characterized in that the solution is prepared by first adding citric acid to water, then dissolving the chitosan, and then adding zinc oxide and lawsone.

3. The method according to claim 1 or 2 characterized in that the solution is prepared by dissolving chitosan in an aqueous solution of citric acid at a temperature of at least 40° C., but the zinc oxide and lawsone are introduced at a temperature of at least 50° C., and after each component is introduced, the solution is mixed intensive and homogenized.

4. The method according to claim 1 or 3 characterized in that the solution is applied to a non-woven fabric by immersing it in the prepared solution at a temperature of at least 30° C. for at least 15 min.

5. The method according to claim 4 characterized in that the nonwoven fabric is dried at a temperature of at least 40° C. for at least 20 min.

6. The method according to claim 1 characterized in that the solution of 0.01-5 wt % essential oils of plant origin is added, especially mint or lavender, which are introduced as the final components of the solution before being applied to the non-woven fabric.
